(19) United States
(12) Patent Application Publication
(10) Pub. No.: US 2026/0253209 A1
(43) Pub. Date: Aug. 27, 2026

(54) MACHINE LEARNING DETECTION OF EYE CONDITIONS

(71) Applicant: HADASIT MEDICAL RESEARCH SERVICES & DEVELOPMENT LTD., Jerusalem (IL)

(72) Inventors: Itay CHOWERS, Moshav Beit Zait (IL); Tomer BATASH, Rishon Letsion (IL); Shlomit BARDUGO, Jerusalem (IL)

(21) Appl. No.: 18/877,149

(22) PCT Filed: Jun. 20, 2023
(86) PCT No.: PCT/IL2023/050638

§ 371 Date: Dec. 19, 2024

Related U.S. Application Data
(60) Provisional application No. 63/353,714, filed on Jun. 20, 2022.

(51) Int. Cl. G06T 7/00; A61B 3/14; G06V 10/774; G06V 10/82; G06V 20/70; G06V 40/18; G16H 50/20

(52) U.S. Cl. CPC G06T 7/0012; G06V 10/774; G06V 10/82; G06V 20/70; G06V 40/193; G16H 50/20; A61B 3/14; G06T 2207/20081; G06T 2207/20084; G06T 2207/30041; G06V 2201/03

(57) ABSTRACT

A method comprising: receiving a set of eye images of a cohort of subjects, and a plurality of datapoints with respect to each of the subjects in the cohort, wherein at least some of the subjects in the cohort of are associated with a diagnosis of a specific eye condition; extracting, with respect to each of the subjects, a set of features representing the images and the datapoints associated with the subject; at a training stage, training a machine learning model on a training dataset comprising: (i) all of the sets of features, and (ii) labels indicating a diagnosis of an eye condition associated with each of the subjects; and at an inference stage, applying the trained machine learning model to a target dataset comprising one or more images and associated datapoints with respect to a target subject, to output a diagnosis of an eye condition in the target subject.

### CROSS-REFERENCE TO RELATED APPLICATIONS

This application claims priority from U.S. Application Ser. No. 63/353,714, filed Jun. 20, 2022, entitled “MACHINE LEARNING DETECTION OF EYE CONDITIONS,” the contents of which are hereby incorporated herein in their entirety by reference.

### FIELD OF THE INVENTION

The present invention relates to the field of machine learning.

### BACKGROUND

Telemedicine eyecare has seen significant development in recent years. Several healthcare providers offer remote-based eye exams and optometry services. In some cases, tests for Glaucoma, Diabetic Retinopathy, AMD, and other posterior segment diseases of the eye may be conducted at primary care sites, wherein the information may then be provided to a remotely-based specialist, for diagnosis and treatment recommendations.

However, much of the progress in the field of eyecare through telemedicine has been focused on posterior segment eye conditions, while less development has been made with respect to anterior segment eye conditions. Anterior segment conditions may be relatively straightforward to diagnose through a simple superficial eye examination, without requiring complex and costly medical or imaging systems. Thus, detecting and diagnosing these conditions by teleophthalmology may be achieved by using common widely-available imaging modalities, such as smartphone cameras, in combination with relevant information about the particulars and history of the patient.

Many primary clinics do not possess the necessary equipment to detect and diagnose eye conditions, and specialist clinics are not readily accessible to many patients. This affects the ability of the medical system to provide patients with effective eye care. This difficulty is exemplified in field medical care facilities (such as in the military), where ocular trauma and disease are common and personnel have limited access to ophthalmic care. It has been reported that the incidence of combat ocular trauma among US forces during recent armed conflicts has been 10-15% of all battle injuries. Disease and non-combat injury are also a common cause of ocular morbidity among deployed service members, with a reported rate of 3.35% per year.

Under field conditions, physicians often use smartphone images taken on site to consult with remotely-based ophthalmologists. However, such informal imaging and data gathering is not performed in an organized or structured manner, and may not comply with privacy and other applicable regulatory requirements.

The foregoing examples of the related art and limitations related therewith are intended to be illustrative and not exclusive. Other limitations of the related art will become apparent to those of skill in the art upon a reading of the specification and a study of the figures.

### SUMMARY OF INVENTION

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive, as input, a set of images of eyes of a cohort of subjects, and a plurality of associated datapoints with respect to each of the subjects in the cohort, wherein at least some of the subjects in the cohort are associated with a diagnosis of a specific eye condition, extract, with respect to each of the subjects, a set of features representing the images and the datapoints associated with the subject, at a training stage, train a machine learning model on a training dataset comprising: (i) all of the sets of features, and (ii) labels indicating a diagnosis of an eye condition associated with each of the subjects, and at an inference stage, apply the trained machine learning model to a target dataset comprising one or more images and associated datapoints with respect to a target subject, to output a diagnosis of an eye condition in the target subject.

There is also provided, in an embodiment, a computer-implemented method comprising: receiving, as input, a set of images of eyes of a cohort of subjects, and a plurality of associated datapoints with respect to each of the subjects in the cohort, wherein at least some of the subjects in the cohort are associated with a diagnosis of a specific eye condition; extracting, with respect to each of the subjects, a set of features representing the images and the datapoints associated with the subject; at a training stage, training a machine learning model on a training dataset comprising: (i) all of the sets of features, and (ii) labels indicating a diagnosis of an eye condition associated with each of the subjects; and at an inference stage, applying the trained machine learning model to a target dataset comprising one or more images and associated datapoints with respect to a target subject, to output a diagnosis of an eye condition in the target subject.

There is further provided, in an embodiment, a computer program product comprising a non-transitory computer-readable storage medium having program instructions embodied therewith, the program instructions executable by at least one hardware processor to: receive, as input, a set of images of eyes of a cohort of subjects, and a plurality of associated datapoints with respect to each of the subjects in the cohort, wherein at least some of the subjects in the cohort are associated with a diagnosis of a specific eye condition; extract, with respect to each of the subjects, a set of features representing the images and the datapoints associated with the subject; at a training stage, train a machine learning model on a training dataset comprising: (i) all of the sets of features, and (ii) labels indicating a diagnosis of an eye condition associated with each of the subjects; and at an inference stage, apply the trained machine learning model to a target dataset comprising one or more images and associated datapoints with respect to a target subject, to output a diagnosis of an eye condition in the target subject.

In some embodiments, the specific eye condition is an anterior segment eye condition.

In some embodiments, the images are annotated to indicate anatomical and pathological eye features represented in the images.

In some embodiments, the annotations are in the form of one of: an exact outline of the anatomical and pathological features, or a bounding box enclosing the anatomical and pathological features.

In some embodiments, the datapoints comprise, with respect to each of the subjects, at least one of the following categories of datapoints: (i) demographic information datapoints; (ii) medical history datapoints; and (iii) eye condition signs and symptoms datapoints.

In some embodiments, the labels represent, with respect to each of the subjects, binary values indicating the presence or absence of an anterior segment eye condition, wherein the diagnosis is expressed as a binary value indicating the presence or absence of an anterior segment eye condition in the target subject.

In some embodiments, the labels represent, with respect to each of the subjects, values on a scale indicating a severity level associated with an anterior segment eye condition, wherein the diagnosis is expressed as a value on a scale indicating a severity level associated with an anterior segment eye condition in the target subject.

In some embodiments, the labels represent, with respect to each of the subjects, a particular anterior segment eye condition selected from a defined set of possible anterior segment eye conditions, wherein the diagnosis indicates a particular anterior segment eye condition in the target subject selected from a defined set of possible anterior segment eye conditions.

In some embodiments, the diagnosis is associated with a confidence score which represents the likelihood that the prediction is correct.

There is further provided, in an embodiment, a system comprising at least one hardware processor; and a non-transitory computer-readable storage medium having stored thereon program instructions, the program instructions executable by the at least one hardware processor to: receive, as input, a set of images of eyes of a cohort of subjects, and a plurality of associated datapoints with respect to each of the subjects in the cohort, wherein at least some of the subjects in the cohort are associated with a diagnosis of a specific eye condition, extract, with respect to each of the subjects, a set of features representing the images and the datapoints associated with the subject, at a training stage, train a machine learning model on a training dataset comprising: (i) all of the sets of features, and (ii) labels indicating a treatment urgency level associated with each of the subjects, and at an inference stage, apply the trained machine learning model to a target dataset comprising one or more eye images and associated datapoints with respect to a target subject, to predict a treatment urgency level associated with the target subject.

There is further provided, in an embodiment, a computer-implemented method comprising: receiving, as input, a set of images of eyes of a cohort of subjects, and a plurality of associated datapoints with respect to each of the subjects in the cohort, wherein at least some of the subjects in the cohort are associated with a diagnosis of a specific eye condition; extracting, with respect to each of the subjects, a set of features representing the images and the datapoints associated with the subject; at a training stage, training a machine learning model on a training dataset comprising: (i) all of the sets of features, and (ii) labels indicating a treatment urgency level associated with each of the subjects; and at an inference stage, applying the trained machine learning model to a target dataset comprising one or more eye images and associated datapoints with respect to a target subject, to predict a treatment urgency level associated with the target subject.

There is further provided, in an embodiment, a computer program product comprising a non-transitory computer-readable storage medium having program instructions embodied therewith, the program instructions executable by at least one hardware processor to: receive, as input, a set of images of eyes of a cohort of subjects, and a plurality of associated datapoints with respect to each of the subjects in the cohort, wherein at least some of the subjects in the cohort are associated with a diagnosis of a specific eye condition; extract, with respect to each of the subjects, a set of features representing the images and the datapoints associated with the subject; at a training stage, train a machine learning model on a training dataset comprising: (i) all of the sets of features, and (ii) labels indicating a treatment urgency level associated with each of the subject; and at an inference stage, apply the trained machine learning model to a target dataset comprising one or more eye images and associated datapoints with respect to a target subject, to predict a treatment urgency level associated with the target subject.

In some embodiments, the specific eye condition is an anterior segment eye condition.

In some embodiments, the images are annotated to indicate anatomical and pathological eye features represented in the images.

In some embodiments, the annotations are in the form of one of: an exact outline of the anatomical and pathological features, or a bounding box enclosing the anatomical and pathological features.

In some embodiments, the datapoints comprise, with respect to each of the subjects, at least one of the following categories of datapoints: (i) demographic information datapoints; (ii) medical history datapoints; and (iii) eye condition signs and symptoms datapoints.

In some embodiments, the predicted treatment urgency level is selected from the group consisting of: urgent treatment required, scheduled treatment required, and follow-up monitoring recommended.

In some embodiments, the prediction is associated with a confidence score which represents the likelihood that the prediction is correct.

In addition to the exemplary aspects and embodiments described above, further aspects and embodiments will become apparent by reference to the figures and by study of the following detailed description.

### BRIEF DESCRIPTION OF THE FIGURES

FIG. 1 is a block diagram of an exemplary system which provides for a software application which enables triage and diagnosis of eye conditions using remote ophthalmology, as supported by a purpose-trained machine learning model, in accordance with some embodiments of the present invention;

FIG. 2A illustrates the functional steps in a method for training a machine learning model configured to predict a triage status (i.e., treatment urgency level) in a subject with a potential diagnosis of anterior segment eye condition or disease, based on remote ophthalmology images and associated subject-specific data, in accordance with some embodiments of the present invention;

FIG. 2B illustrates the functional steps in a method for training a machine learning model configured predicting an anterior segment eye condition or disease in a subject, based on remote ophthalmology images and associated subject-specific data, according to some embodiments of the present disclosure;

FIGS. 3A-3D schematically illustrate the process of inferencing a machine learning model of the present technique, according to some embodiments of the present disclosure; and FIGS. 4A-4H illustrate an exemplary user interface of a software application for remote data gathering from a target subject, according to some embodiments of the present disclosure.

### DETAILED DESCRIPTION

Disclosed herein is a technique, embodies in a system, computer-implemented method, and computer program product, which provides for triage and diagnosis of eye conditions using remote ophthalmology, as supported by a purpose-trained machine learning model.

In some embodiments, the present technique may be realized in a software application comprising a machine learning model, configured for carrying out remote medical diagnosis of ophthalmic diseases and conditions. In some embodiments, the present software application may be configured for execution on a mobile device, such as a smartphone or tablet computer.

In some embodiments, the software application of the present technique includes a patient interface for patient-specific data gathering. In some embodiments, the patient interface is configured to guide the patient through a structured questionnaire to gather demographic information about the patient, as well as self-reported symptoms. In some embodiments, the patient interface is further configured to guide the patient through one or more standardized visual acuity tests. In some embodiments, the patient interface further includes a facility for guiding the patient through acquiring and uploading one or more images of the patient's eye, e.g., using a camera of the mobile device or any similar digital camera.

In one exemplary implementation, a software application of the present technique may be executed on a mobile device or another home device. The software application may provide for a structured questionnaire for self-completion by the patient, to gather demographic information about the patient, as well as self-reported symptoms and self-obtained images of the eye. In some embodiments, the questionnaire and other data gathering steps may be designed for self-completion without any supervision by a medical practitioner. In other cases, a medical practitioner, such as an ophthalmologist, may provide online real-time, near-real time, or non-direct quality control and guidance to the patient, by reviewing questionnaire responses and/or other data gathering steps, and providing direct feedback. Post-completion, the ophthalmologist may review the submitted questionnaire responses and/or other data provided, and provide diagnosis and treatment recommendations.

In another exemplary implementation, a software application of the present technique may be executed on a dedicated device, e.g., a dedicated ophthalmic healthcare system. The software application may provide for a structured questionnaire for self-completion by the patient, to gather demographic information about the patient, as well as self-reported symptoms and self-obtained images of the eye. In some embodiments, the questionnaire and other data gathering steps may be designed for self-completion without any real-time supervision by a medical practitioner. In other cases, a medical practitioner, such as an ophthalmologist, may provide online real-time, near-real time, or non-direct, quality control and guidance to the patient, by reviewing questionnaire responses and/or other data gathering steps, and providing direct feedback. Post-completion, an ophthalmologist may review the submitted questionnaire responses and/or other data provided, and provide diagnosis and treatment recommendations.

In yet another exemplary implementation, a software application of the present technique may be executed on a mobile device or another home device. The software application may provide for at least partially guided-completion, in the presence and under the supervision of a medical practitioner, such as a healthcare worker, which provides real-time quality control and guidance to the patient. In some cases, an ophthalmologist may also provide online real-time, near-real time, or non-direct quality control and guidance to the patient, by reviewing questionnaire responses and/or other data gathering steps, and providing direct feedback. Post-completion, an ophthalmologist may review the submitted questionnaire responses and/or other data provided, and provide diagnosis and treatment recommendations.

In a further exemplary implementation, a software application of the present technique may be executed on a mobile device or another home device. The software application may provide for a structured questionnaire for self-completion by the patient, to gather demographic information about the patient, as well as self-reported symptoms and self-obtained images of the eye. In some cases, a medical practitioner, such as a healthcare worker, may provide online real-time, near-real time, or non-direct quality control and guidance to the patient, by reviewing questionnaire responses and/or other data gathering steps, and providing direct feedback. In some cases, an ophthalmologist may provide online real-time, near-real time, or non-direct quality control and guidance to the healthcare worker, by reviewing questionnaire responses and/or other data gathering steps, and providing direct feedback to the healthcare worker. Post-completion, an ophthalmologist may review the submitted questionnaire responses and/or other data provided, and provide diagnosis and treatment recommendations.

In some embodiments, natural language processing (NLP) and artificial intelligence (AI) tools, such as chatbots and the like, may be used for review and quality control of questionnaire responses and other data submitted by a patient. For example, such tools may detect incomplete or non-responsive answers. In some embodiments, NLP and AI tools may further be used for review and quality control of diagnoses and treatment recommendations by ophthalmologists.

In some embodiments, decision trees algorithms may be used to guide a patient through different branches of questions trees and supplemental data requests, for example, based on previous answers by the patient. Likewise, NLP and AI tools may be used to select follow-up questions and prompts, based on previous answers and data provided by the patient.

In some embodiments, the software application of the present technique also includes a clinician interface to facilitate evaluation and analysis of the data and images gathered from the patient, and to provide treatment recommendations. In some embodiments, an output of the clinician evaluation and analysis phase may be one of the following treatment recommendations:
(i) Triage: Referral to an emergency room or an ophthalmologist, referral to schedule a later appointment with an ophthalmologist, or referral for follow up monitoring.
(ii) Diagnosis: Differential diagnosis and leading diagnosis for the condition.
(iii) On-site treatment instructions: Instructions for direct on-site or self-administered treatment steps, such as washing eye, removing contact lenses, using eye drops, and the like.

In some embodiments, the software application of the present technique provides for a secure platform for gathering and sharing medical information about one or more patients, which meets industry and regulatory privacy and data protection requirements. For example, the software application of the present technique may implement information privacy and security standards in compliance with such regulations, such as the U.S. Health Insurance Portability and Accountability Act (HIPAA), Fast Healthcare Interoperability Resources (FHIR) for accessing Electronic Medical Records (EMR), and E.U. General Data Protection Regulation (GDPR).

In some embodiments, the present technique provides for one or more trained machine learning models configured for triage and diagnosis of eye conditions in a patient, based on remote ophthalmology images and associated patient-specific data. In some embodiments, the present technique provides for training one or more machine learning models, to enable automated remote analysis of ophthalmic images, to generate predictions of ophthalmic conditions, and to provide decision support to clinicians. In some embodiments, one or more rained machine learning models of the present technique is configured to analyze clinical data and eye images, and serve as a decision-supporting tool to advise a physician in predicting a triage status (i.e., treatment urgency level) in a subject, making a diagnosis, as well as providing treatment and case management recommendations.

In some embodiments, a first exemplary machine learning model of the present technique may be trained to predict a triage status in a subject with a potential diagnosis of anterior segment eye condition or disease, based on remote ophthalmology images and associated patient-specific data. In some embodiments, the predicted triage status (i.e., a degree of care urgency) in the subject may be one of:
Urgent care: Referral for immediate treatment at an emergency care facility or by an ophthalmologist.
Non-Urgent care: Referral for scheduled treatment at a future date.
No care required: No referral for further treatment required at the time/recommended follow up monitoring.

In some embodiments, the first exemplary machine learning model may be trained on a training dataset comprising a plurality of images of the eyes of a cohort of subjects, as well as a plurality of associated datapoints with respect to each of the subjects in the cohort. In some embodiments, the cohort of subjects includes at least (i) a first subgroup of subjects representing cases with a potential diagnosis of anterior segment eye condition or disease, and (ii) a second subgroup of subjects representing cases with no potential diagnosis of anterior segment eye condition or disease.

In some embodiments, a second exemplary machine learning model of the present technique may be trained to predict an anterior segment eye condition or disease in a subject, based on remote ophthalmology images and associated patient-specific data.

In some embodiments, the second exemplary machine learning model may be trained on a training dataset comprising a plurality of images of the eyes of a cohort of subjects, as well as a plurality of associated datapoints with respect to each of the subjects in the cohort. In some embodiments, the cohort of subjects includes at least (i) a first subgroup of subjects representing cases with a potential diagnosis of anterior segment eye condition or disease, and (ii) a second subgroup of subjects representing cases with no potential diagnosis of anterior segment eye condition or disease.

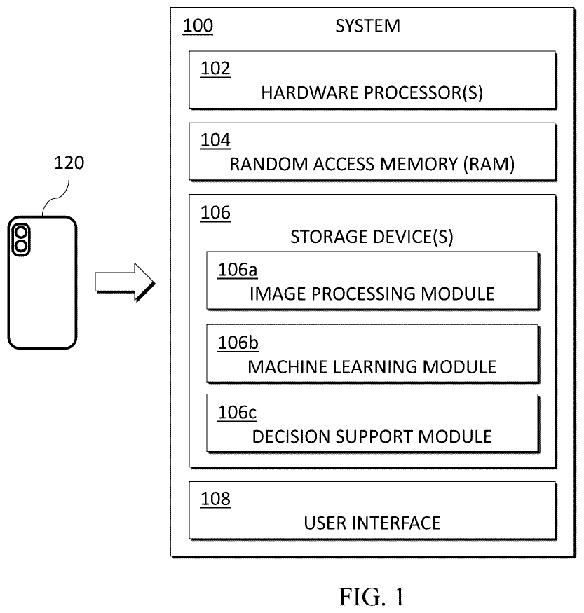

Reference is made to FIG. 1, which is a block diagram of an exemplary system 100 which provides for a software application which enables triage and diagnosis of eye conditions using remote ophthalmology, as supported by a purpose-trained machine learning model, in accordance with some embodiments of the present invention.

In some embodiments, system 100 may comprise a hardware processor 102, and a random-access memory (RAM) 104, and/or one or more non-transitory computer-readable storage device 106. In some embodiments, system 100 may store in storage device 106 software instructions or components configured to operate a processing unit (also ‘hardware processor,’ ‘CPU,’ ‘quantum computer processor,’ or simply ‘processor’), such as hardware processor 102. In some embodiments, the software components may include an operating system, including various software components and/or drivers for controlling and managing general system tasks (e.g., memory management, storage device control, power management, etc.) and facilitating communication between various hardware and software components. Components of system 100 may be co-located or distributed, or the system may be configured to run as one or more cloud computing ‘instances,’ ‘containers,’ ‘virtual machines,’ or other types of encapsulated software applications, as known in the art.

The software instructions and/or components operating hardware processor 102 may include instructions for receiving and analyzing multiple scan slices captured by any suitable volumetric imaging system. For example, hardware processor 102 may comprise an image processing module 106a, a machine learning module 106b, and a decision support module 106c.

In some embodiments, image processing module 106a receives one or more images of an eye of a patient of interest, acquired using, e.g., a mobile device such as smartphone 120. In some embodiments, image processing module 106a applies one or more image processing algorithms thereto. In some embodiments, image processing module 106a comprises one or more algorithms configured to perform object detection, classification, segmentation, and/or any other similar operation, using any suitable image processing, algorithm technique, and/or feature extraction process. The input images may come from various imaging devices having varying settings, configuration and/or scan acquisition parameters. Depending on the embodiment, the image processing module 106a can route scans through various processing functions, or to an output circuit that sends the processed scans for presentation, e.g., on a display, to a recording system, across a network, a remote or cloud server, or to any other logical destination. The image processing module 106a may apply scan processing algorithms alone or in combination. Image processing module 106a may also facilitate logging or recording operations with respect to an input image.

Machine learning module 106b may comprise any one or more neural networks (i.e., which include one or more neural network layers), and can be implemented to embody any appropriate neural network architecture, e.g., U-Net, Mask R-CNN, DeepLab, and the like. In a particular example, machine learning module 106b may include an input layer followed by a sequence of shared convolutional neural network layers. The output of the final shared convolutional neural network layer may be provided to a sequence of one or more additional neural network layers that are configured to generate the output. However, other appropriate neural network processes may also be used, however. The output of the final shared convolutional neural network layers may be provided to a different sequence of one or more additional neural network layers.

Decision support module 106c may comprise one or more algorithms for formulating a diagnosis, as well as providing treatment and case management recommendations.

In some embodiments, system 100 may further comprise a user interface 108 comprising, e.g., a display monitor for displaying images, a control panel for controlling system 100, and a speaker for providing audio feedback.

System 100 as described herein is only an exemplary embodiment of the present invention, and in practice may be implemented in hardware only, software only, or a combination of both hardware and software. System 100 may have more or fewer components and modules than shown, may combine two or more of the components, or may have a different configuration or arrangement of the components. System 100 may include any additional component enabling it to function as an operable computer system, such as a motherboard, data busses, power supply, a network interface card, a display, an input device (e.g., keyboard, pointing device, touch-sensitive display), etc. (not shown).

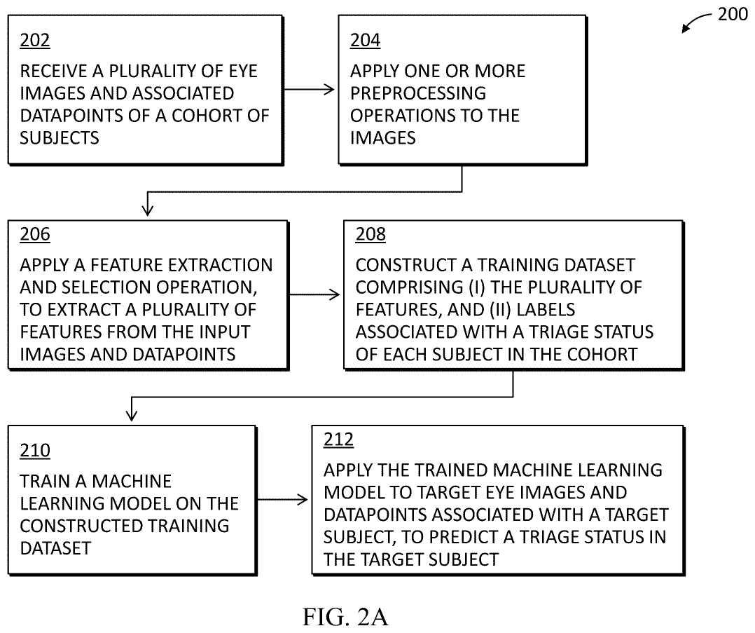

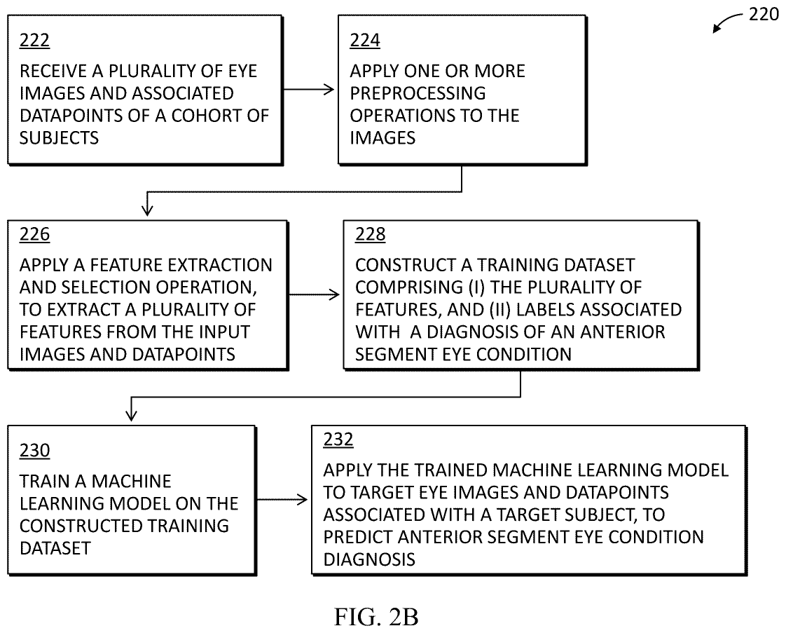

The instructions of system 100 will now be discussed with reference to the flowchart of FIG. 2A, which illustrates the functional steps in a method 200 for training a machine learning model configured to predict a triage status (i.e., treatment urgency level) in a subject with a potential diagnosis of anterior segment eye condition or disease, based on remote ophthalmology images and associated subject-specific data. In some embodiments, the predicted triage status in the subject may be one of:
Urgent care: Referral for immediate treatment at an emergency care facility or by an ophthalmologist.
Non-Urgent care: Referral for scheduled treatment at a future date.
No care required: No referral for further treatment required at the time/recommended follow up monitoring.

The various steps of method 200 will be described with continuous reference to exemplary system 100 shown in FIG. 1. The various steps of method 200 may either be performed in the order they are presented or in a different order (or even in parallel), as long as the order allows for a necessary input to a certain step to be obtained from an output of an earlier step. In addition, the steps of method 200 may be performed automatically (e.g., by system 100 of FIG. 1), unless specifically stated otherwise. In addition, the steps of method 200 are set forth for exemplary purposes, and it is expected that modification to the flow chart is normally required to accommodate various network configurations and network carrier business policies.

Method 200 begins in step 202, wherein system 100 may receive, as input, a database comprising a plurality of images of the eyes of a cohort of subjects, as well as a plurality of associated datapoints with respect to each of the subjects in the cohort. In some embodiments, the cohort of subjects includes at least (i) a first subgroup of subjects associated with a potential diagnosis of anterior segment eye condition or disease, and (ii) a second subgroup of subjects representing cases with no potential diagnosis of anterior segment eye condition or disease.

In some embodiments, the images may comprise, at least in part, images of the anterior segment of the eyes acquired using any suitable imaging device, such as a mobile device camera or an equivalent device. In some embodiments, at least some of the images include annotations associated with anatomical and pathological features represented in the images. In some embodiments, the annotations are in the form of an exact outline of the anatomical and pathological features, or a bounding box enclosing the anatomical and pathological features. In some embodiments, the images may be annotated and/or labelled manually, e.g., by specialists, and/or using any other method of annotation.

In some embodiments, such annotations may indicate, but are not limited to:
Hyphema: Traumatic, iatrogenic (e.g., intraocular surgery or laser), iris neovascularization, herpes simplex or zoster iridocyclitis, blood dyscrasia or clotting disorder (e.g., hemophilia), anticoagulation, Fuchs heterochromic iridocyclitis, intraocular tumor (e.g., juvenile xanthogranuloma, retinoblastoma, angioma).
Hypopyon: Infectious corneal ulcer, endophthalmitis, severe iridocyclitis (e.g., HLA-B27 associated, Behcet disease), reaction to an intraocular lens (sterile hypopyon), retained lens particle, device contaminant after cataract surgery (toxic anterior segment syndrome), intraocular tumor necrosis (e.g., pseudohypopyon from retinoblastoma), retained intraocular foreign body, tight contact lens, chronic corneal edema with ruptured bullae, severe inflammatory reaction from a recurrent corneal erosion, drugs (e.g., rifampin).
Conjunctival Swelling (Chemosis): Allergy, any ocular or periocular inflammation, postoperative, drugs, venous congestion (e.g., c-c fistula), angioneurotic edema, myxedema.
Conjunctival Dryness (Xerosis): Vitamin A deficiency, post-cicatricial conjunctivitis, Stevens-Johnson syndrome, ocular cicatricial pemphigoid, exposure (e.g., lagophthalmos, absent blink reflex, proptosis), radiation, chronic dacryoadenitis, Sjogren syndrome.
Corneal Edema:
   Congenital: Congenital glaucoma, congenital hereditary endothelial dystrophy, posterior polymorphous corneal dystrophy (PPMD), birth trauma (forceps injury).
   Acquired: Postoperative edema, aphakic or pseudophakic bullous keratopathy, Fuchs endothelial dystrophy, contact lens overwear, traumatic, exposure-related, chemical injury, acute increase in intraocular pressure (e.g., angle-closure glaucoma), corneal hydrops (decompensated keratoconus), herpes simplex or zoster keratitis, iritis, failed corneal graft, iridocorneal endothelial (ICE) syndrome, PPMD.
Dilated Episcleral Vessels (Without Ocular Irritation or Pain): Underlying uveal neoplasm, arteriovenous fistula (e.g., c-c fistula), polycythemia vera, leukemia, ophthalmic vein or cavernous sinus thrombosis, extravascular blockage of ophthalmic/orbital venous outflow.
Enlarged Corneal Nerves: Multiple endocrine neoplasia type IIb (medullary carcinoma of the thyroid gland, pheochromocytoma, mucosal neuromas; may have marfanoid habitus), acanthamoeba keratitis, chronic keratitis, keratoconus, neurofibromatosis, Fuchs endothelial dystrophy, Refsum syndrome, trauma, congenital glaucoma, failed corneal graft, leprosy, ichthyosis, idiopathic, normal variant.
Follicles on the Conjunctiva: Acute Conjunctivitis and chronic conjunctivitis.
Membranous Conjunctivitis: Streptococci, pneumococci, chemical burn, ligneous conjunctivitis, Corynebacterium diphtheriae, herpes simplex virus, ocular vaccinia.
Pseudomembranous Conjunctivitis: Ocular cicatricial pemphigoid, Stevens-Johnson syndrome, superior limbic keratoconjunctivitis, gonococci, staphylococci, chlamydia in newborns, and others.
Opacification of the Cornea in Infancy: Congenital glaucoma, birth trauma (forceps injury), congenital hereditary endothelial or stromal dystrophy (bilateral), PPMD, developmental abnormality of the anterior segment (e.g., Peters anomaly), metabolic abnormalities (bilateral; e.g., mucopolysaccharidoses, mucolipidoses), interstitial keratitis, herpes simplex virus, corneal ulcer, corneal dermoid, sclerocornea.
Pannus (Superficial Vascular Invasion of the Cornea): Ocular rosacea, tight contact lens or contact lens overwear, phlyctenule, chlamydia (trachoma and inclusion conjunctivitis), superior limbic keratoconjunctivitis (micropannus only), staphylococcal hypersensitivity, vernal keratoconjunctivitis, herpes simplex or zoster virus, chemical burn, ocular cicatricial pemphigoid, aniridia, molluscum contagiosum, leprosy.
Papillae on the Conjunctiva: Acute conjunctivitis and chronic conjunctivitis
Pigmentation/Discoloration of the Conjunctiva: Racial melanosis (perilimbal), nevus, primary acquired melanosis, melanoma, ocular and oculodermal melanocytosis (congenital, blue-gray, not conjunctival but episcleral), Addison disease, pregnancy, radiation, jaundice, resolving subconjunctival hemorrhage, conjunctival or subconjunctival foreign body, pharmacologic (e.g., chlorpromazine, topical epinephrine), cosmetic (e.g., mascara/makeup deposits, tattoo).
Symblepharon (Fusion of the Palpebral Conjunctiva with the Bulbar Conjunctiva): Ocular cicatricial pemphigoid, Stevens-Johnson syndrome, chemical burn, trauma, drugs, long-standing conjunctival or episcleral inflammation, epidemic keratoconjunctivitis, atopic conjunctivitis, radiation, congenital, iatrogenic (postsurgical).
Eyelid Abnormalities.
Eyelid edema.
Eyelid lesion (benign or malignant lesions).
Ptosis and Pseudoptosis.
Iris Heterochromia (Irises of Different Colors):
   Involved iris is lighter than normal: Congenital Horner syndrome, most cases of Fuchs heterochromic iridocyclitis, chronic uveitis, juvenile xanthogranuloma, metastatic carcinoma, Waardenburg syndrome.
   Involved iris is darker than normal: Ocular melanocytosis or oculodermal melanocytosis, hemosiderosis, siderosis, retained intraocular foreign body, ocular malignant melanoma, diffuse iris nevus, retinoblastoma, leukemia, lymphoma, ICE syndrome, some cases of Fuchs heterochromic iridocyclitis.
Iris Lesion:
   Melanotic (brown): Nevus, melanoma, adenoma, or adenocarcinoma of the iris pigment epithelium.
   Amelanotic (white, yellow, or orange): Amelanotic melanoma, inflammatory nodule or granuloma (e.g., sarcoidosis, tuberculosis, leprosy, other granulomatous disease), neurofibroma, patchy hyperemia of syphilis, juvenile xanthogranuloma, medulloepithelioma, foreign body, cyst, leiomyoma, seeding from a posterior segment tumor.
Neovascularization of the Iris: Diabetic retinopathy, ocular ischemic syndrome, after central or branch retinal vein or artery occlusion, chronic uveitis, chronic retinal detachment, intraocular tumor (e.g., retinoblastoma, melanoma), other retinal vascular disease.
Dislocated Lens (Ectopia Lentis).
Iridescent Lens Particles: Drugs, hypocalcemia, myotonic dystrophy, hypothyroidism, familial, idiopathic
Lenticonus:
   Anterior (marked convexity of the anterior lens): Alport syndrome (hereditary nephritis).
   Posterior (marked concavity of the posterior lens surface): Usually idiopathic, may be associated with persistent fetal vasculature.
Afferent Pupillary Defect: Optic nerve disease (e.g., ischemic optic neuropathy, optic neuritis, tumor, glaucoma); central retinal artery or vein occlusion; less commonly, a lesion of the optic chiasm or tract, Any of the preceding, amblyopia, dense vitreous hemorrhage, advanced macular degeneration, branch retinal vein or artery occlusion, retinal detachment, or other retinal disease.
Anisocoria (Pupils of Different Sizes).
Limitation of Ocular Motility:
   With exophthalmos and resistance to retropulsion: Orbital Disease
   Without exophthalmos and resistance to retropulsion: Isolated third, fourth, or sixth cranial nerve palsy; multiple ocular motor nerve palsies, CAVERNOUS SINUS AND ASSOCIATED SYNDROMES, myasthenia gravis, chronic progressive external ophthalmoplegia and associated syndromes, orbital blow-out fracture with muscle entrapment, ophthalmoplegic migraine, Duane syndrome, other central nervous system (CNS) disorders.
Paradoxical Pupillary Reaction (Pupil Dilates in Light and Constricts in Darkness): Congenital stationary night blindness, congenital achromatopsia, optic nerve hypoplasia, Leber congenital amaurosis, Best disease, optic neuritis, dominant optic atrophy, albinism, retinitis pigmentosa. Rarely amblyopia
Extraocular Muscle Thickening: Thyroid orbitopathy (often spares tendon), idiopathic orbital inflammatory syndrome, tumor (e.g., lymphoma, metastasis, or spread of lacrimal gland tumor to muscle), c-c fistula, superior ophthalmic vein thrombosis, cavernous hemangioma (usually appears in the muscle cone without muscle thickening), rhabdomyosarcoma (children). Lacrimal Gland Lesions Orbital Lesions/Proptosis.

In some embodiments, the input datapoints received in step 202 may include, with respect to at least some of the subjects in the cohort, personal and/or demographic information, including, but not limited to:
Age,
sex (male/female),
ethnicity,
occupation,
general medical history,
history of ocular/eye trauma,
history of ocular/eye conditions or diseases, use of corrective lenses, and/or
use of contact lenses.

In some embodiments, the input datapoints received in step 202 may include, with respect to at least some of the subjects in the cohort, measures, signs (i.e., objective and externally observable conditions) and symptoms (i.e., subjective reported experiences), associated with eye conditions, injuries, and/or diseases in each such subject, including, but not limited to:
Identity of eye associated with the symptoms (left/right),
visual acuity, or the ability of the eye to distinguish shapes and the details of objects at a given distance,
burning sensation in the eye,
decreased vision,
transient loss of vision and duration of loss,
loss of visons for longer than 24 hours:
   sudden loss,
   gradual loss,
   painful loss,
post-traumatic loss of vision,
eye discharge,
distortion of vision,
double vision (monocular or binocular),
dry eye,
eyelash loss,
eyelid crusting,
eyelid drooping,
eyelid swelling (inflammatory or non-inflammatory),
eyelid twitching,
eyelid unable to close (lagophthalmos),
eyelid bulging (proptosis),
eyelid jumping,
flashes of light,
foreign body sensation,
glare,
halos around lights,
itchy eyes,
light sensitivity (photophobia),
night blindness,
ocular pain,
periorbital pain,
redeye,
spots in front of eyes (transient, long lasting, or permanent), and/or
excessive tearing.
Leukocoria (White Pupillary Reflex).
Nystagmus in Infancy: Congenital nystagmus, albinism, Leber congenital amaurosis, CNS (thalamic) injury, spasmus nutans, optic nerve or chiasmal glioma, optic nerve hypoplasia, congenital cataracts, aniridia, congenital corneal opacities.
Shallow Anterior Chamber:
   Accompanied by increased intraocular pressure: Pupillary block glaucoma, capsular block syndrome, suprachoroidal hemorrhage, malignant glaucoma.
   Accompanied by decreased intraocular pressure: Wound leak, choroidal detachment, over-filtration after glaucoma filtering procedure.
   Hypotony: Wound leak, choroidal detachment, cyclodialysis cleft, retinal detachment, ciliary body shutdown, pharmacologic aqueous suppression, over-filtration after glaucoma filtering procedure.
Progressive Hyperopia: Orbital tumor pressing on the posterior surface of the eye, serous elevation of the retina (e.g., central serous chorioretinopathy), posterior scleritis, presbyopia, hypoglycemia, cataracts, after radial keratotomy or other refractive surgery.
Progressive Myopia: High (pathologic) myopia, diabetes, cataract, staphyloma and elongation of the globe, corneal ectasia (keratoconus or after corneal refractive surgery), medications (e.g., miotic drops, sulfa drugs, tetracycline), childhood (physiologic).

In some embodiments, in step 204, a data preprocessing stage may take place, comprising at least one of data cleaning and normalizing, removal of missing data, data quality control, and/or any other suitable preprocessing method or technique.

In some embodiments, in step 206, a feature extraction and selection stage may be performed. In some embodiments, feature extraction includes the generation of a feature set with respect to each of the subjects in the cohort, based on analysis and processing of the input images and associated datapoints associated with each subject. In some embodiments, the extracted features are derived in a feature space using predefined processes for each data type of the input data received in step 202, including the input images and associated datapoints.

Feature selection may involve one or more feature selection operations (e.g., feature selection, parameter selection), to reduce the number of features and variables to identify a subset of features or variables in the input data which have desired predictive ability relative to other features in the input data.

In some embodiments, in step 208, the present technique provides for constructing a training dataset comprising:
(i) The set of features extracted in step 206 with respect to each subject in the cohort of subjects, and
(ii) labels associated with a triage status (i.e., a degree of care urgency) in each of the subjects.

In some embodiments, the labels may be determined by experienced specialists, such as ophthalmologists, with respect to each subject in the cohort of subjects.

In some embodiments, in step 210, a machine learning model of the present technique may be trained on the training dataset constructed in step 208. In some embodiments. In some embodiments, the machine learning model may comprise any one or more neural networks (i.e., which include one or more neural network layers), and can be implemented to embody any appropriate neural network architecture, e.g., U-Net, Mask R-CNN, DeepLab, and the like.

In some embodiments, a trained machine learning model of the present technique may be configured to receive, as input, a target dataset comprising one or more images and associated datapoints with respect to a target subject, and to predict a triage status (i.e., treatment urgency level) in a subject with a potential diagnosis of anterior segment eye condition or disease. In some embodiments, the predicted triage status in the subject may be one of:
Urgent care: Referral for immediate treatment at an emergency care facility or by an ophthalmologist.
Non-Urgent care: Referral for scheduled treatment at a future date.
No care required: No referral for further treatment required at the time/recommended follow up monitoring.

In some embodiments, the trained machine learning model may be further configured to assign a confidence score to each prediction which represents the likelihood that the prediction is correct.

In some embodiments, the trained machine learning model may be configured to predict a triage status on a severity scale. In such cases, the labels assigned to the feature sets within the training dataset may be represented as a scale of, e.g., 1-3 or 1-5. In some embodiments, the scale may include two or more discrete categories such as ‘refer to emergency treatment,’ ‘follow-up treatment,’ and ‘non-emergency treatment.’

In some embodiments, at an inference step 212, the trained machine learning model of the present technique may be applied to a target dataset comprising one or more eye images and associated datapoints with respect to a target subject, to predict a triage status in the target subject. In some embodiments, the machine learning model may be further configured to assign a confidence score to each prediction which represents the likelihood that the prediction is correct.

In some embodiments, the target dataset may be acquired, at least in part, from a target subject at a remote location, using, e.g., a software application running on a mobile device, such as smartphone 120 shown in FIG. 1.

In some embodiments, inference step 212 may be implemented using a software application of the present technique, which may be executed on a mobile device or another home device. The software application may provide for a structured questionnaire for self-completion by the target subject, to gather the target eye images and associated datapoints. A medical practitioner, such as an ophthalmologist, may provide online real-time, near-real time, or non-direct quality control and guidance to the patient, by reviewing questionnaire responses and/or other data gathering steps, and providing direct feedback.

In another exemplary implementation, inference step 212 may be implemented using a software application of the present technique, which may be executed on a dedicated device, e.g., a dedicated ophthalmic healthcare system. The software application may provide for a structured questionnaire for self-completion by the target subject, to gather the target eye images and associated datapoints. A medical practitioner, such as an ophthalmologist, may provide online real-time, near-real time, or non-direct, quality control and guidance to the patient, by reviewing questionnaire responses and/or other data gathering steps, and providing direct feedback.

In yet another exemplary implementation, inference step 212 may be implemented using a software application of the present technique, which may be executed on a mobile device or another home device. The software application may provide for at least partially guided-completion by the target subject, in the presence and under the supervision of a medical practitioner, such as a healthcare worker, which provides real-time quality control and guidance to the target subject. An ophthalmologist may also provide online real-time, near-real time, or non-direct quality control and guidance to the patient, by reviewing questionnaire responses and/or other data gathering steps, and providing direct feedback.

In a further exemplary implementation, inference step 212 may be implemented using a software application of the present technique, which may be executed on a mobile device or another home device. The software application may provide for a structured questionnaire for self-completion by the target subject, to gather the target eye images and associated datapoints. A medical practitioner, such as a healthcare worker, may provide online real-time, near-real time, or non-direct quality control and guidance to the target subject, by reviewing questionnaire responses and/or other data gathering steps, and providing direct feedback. An ophthalmologist may provide online real-time, near-real time, or non-direct quality control and guidance to the healthcare worker, by reviewing questionnaire responses and/or other data gathering steps, and providing direct feedback to the healthcare worker.

The instructions of system 100 will now be further discussed with reference to the flowchart of FIG. 2B, which illustrates the functional steps in a method 220 for training a machine learning model configured for predicting an anterior segment eye condition or disease in a subject, based on remote ophthalmology images and associated subject-specific data.

The various steps of method 220 will be described with continuous reference to exemplary system 100 shown in FIG. 1. The various steps of method 220 may either be performed in the order they are presented or in a different order (or even in parallel), as long as the order allows for a necessary input to a certain step to be obtained from an output of an earlier step. In addition, the steps of method 220 may be performed automatically (e.g., by system 100 of FIG. 1), unless specifically stated otherwise. In addition, the steps of method 220 are set forth for exemplary purposes, and it is expected that modification to the flow chart is normally required to accommodate various network configurations and network carrier business policies.

Method 220 begins in step 222, wherein system 100 may receive, as input, a set of images of the eyes of a cohort of subjects, as well as a plurality of associated datapoints with respect to each of the subjects in the cohort. In some embodiments, the cohort of subjects includes at least (i) a first subgroup of subjects associated with a potential diagnosis of anterior segment eye condition or disease, and (ii) a second subgroup of subjects representing cases with no potential diagnosis of anterior segment eye condition or disease.

In some embodiments, the images may comprise, at least in part, images of the anterior segment of the eyes acquired using any suitable imaging device, such as a mobile device camera or an equivalent device. In some embodiments, at least some of the images include annotations associated with anatomical and pathological features represented in the images. In some embodiments, the annotations are in the form of an exact outline of the anatomical and pathological features, or a bounding box enclosing the anatomical and pathological features. In some embodiments, the images may be annotated and/or labelled manually, e.g., by specialists, and/or using any other method of annotation.

In some embodiments, such annotations may include, but are not limited to, some or all of the annotations detailed with respect to step 202 in method 200 hereinabove.

In some embodiments, the input datapoints received in step 222 may include, with respect to at least some of the subjects in the cohort, personal and/or demographic information, including, but not limited to, some or all of the personal and/or demographic information detailed with respect to step 202 in method 200 hereinabove.

In some embodiments, the input datapoints received in step 222 may include, with respect to at least some of the subjects in the cohort, measures, signs (i.e., objective and externally observable conditions) and symptoms (i.e., subjective reported experiences), associated with eye conditions, injuries, and/or diseases in each such subject, including, but not limited to, some or all of the measures, signs, and symptoms detailed with respect to step 202 in method 200 hereinabove.

In some embodiments, in step 224, a data preprocessing stage may take place, comprising at least one of data cleaning and normalizing, removal of missing data, data quality control, and/or any other suitable preprocessing method or technique.

In some embodiments, in step 226, a feature extraction and selection stage may be performed. In some embodiments, feature extraction includes the generation of a feature set with respect to each of the subjects in the cohort, based on analysis and processing of the input images and associated datapoints associated with each subject. In some embodiments, the extracted features are derived in a feature space using predefined processes for each data type of the input data received in step 222, including the input images and associated datapoints.

Feature selection may involve one or more feature selection operations (e.g., feature selection, parameter selection), to reduce the number of features and variables to identify a subset of features or variables in the input data which have desired predictive ability relative to other features in the input data.

In some embodiments, in step 228, the present technique provides for constructing a training datasets comprising:
(i) The set of features extracted in step 226 with respect to each subject in the cohort of subjects, and
(ii) labels associated with a diagnosis of an anterior segment eye condition or disease associated with each of the subjects.

In some embodiments, the labels may be represented as a binary value, e.g., or [yes/no]. In some embodiments, the labels may be represented as a discrete scale of, e.g., 1-3 or 1-5.

In some embodiments, in step 230, a machine learning model of the present technique may be trained on the training dataset constructed in step 228. In some embodiments. In some embodiments, the machine learning model may comprise any one or more neural networks (i.e., which include one or more neural network layers), and can be implemented to embody any appropriate neural network architecture, e.g., U-Net, Mask R-CNN, DeepLab, and the like.

In some embodiments, the trained machine learning model of the present technique may be configured to receive, as input, a target dataset comprising one or more images and associated datapoints with respect to a target subject, and to predict a diagnosis associated with an anterior segment eye condition in the target subject. In some embodiments, the machine learning model may be further configured to assign a confidence score to each prediction which represents the likelihood that the prediction is correct.

In some embodiments, a trained machine learning model of the present disclosure provides for predicting the presence or absence of an anterior segment eye condition in a target subject. In such cases, the labels assigned to the feature sets within the training dataset may be represented as a binary value, e.g., [1/0] or [yes/no].

In some embodiments, a trained machine learning model of the present disclosure provides for predicting the severity level of an anterior segment eye condition in a target subject, wherein the prediction may be expressed on a scale. For example, a machine learning model of the present disclosure may provide for predicting a severity level of an anterior segment eye condition in a target subject on a severity scale. In such cases, the labels assigned to the feature sets within the training dataset may be represented as a scale of, e.g., 1-3 or 1-5.

In some embodiments, a trained machine learning model of the present disclosure provides for predicting a particular anterior segment eye condition in a target subject, from a defined set of possible anterior segment eye conditions. In such cases, the labels assigned to the feature sets within the training dataset may be represented as a set of anterior segment eye conditions. An exemplary set of anterior segment eye conditions may include, but is not limited to, some of the following diagnoses associated with each of the symptoms listed in step 222 above (as detailed with reference to step 202 of method 200 hereinabove):
Burning sensation: Blepharitis, meibomitis, dry eye syndrome, conjunctivitis (infectious, allergic, mechanical, chemical), corneal defects (usually marked by fluorescein staining of the cornea), inflamed pterygium or pinguecula, episcleritis, superior limbic keratoconjunctivitis, ocular toxicity (medication, makeup, contact lens solutions), contact lens-related problems.
Loss of vision:
   Transient vision loss (vision returns to normal within 24 hours, usually within 1 hour): Papilledema, amaurosis fugax (transient ischemic attack; unilateral), vertebrobasilar artery insufficiency (bilateral), migraine (with or without a subsequent headache), impending central retinal vein occlusion, ischemic optic neuropathy, ocular ischemic syndrome (carotid occlusive disease), glaucoma, sudden change in blood pressure, central nervous system (CNS) lesion, optic disc drusen, orbital lesion (vision loss may be associated with eye movement).
   Vision loss lasting more than 24 hours:
      Sudden, painless loss: Retinal artery or vein occlusion, ischemic optic neuropathy, giant cell arteritis, vitreous hemorrhage, retinal detachment, optic neuritis (pain with eye movement in >50% of cases), sudden discovery of pre-existing unilateral vision loss, other retinal or CNS disease (e.g., stroke), toxins (e.g., methanol poisoning), ophthalmic artery occlusion (may also have extraocular motility deficits and ptosis).
      Gradual, painless loss (over weeks, months, or years): Cataract, refractive error, open angle glaucoma, chronic angle closure glaucoma, chronic retinal disease such as age-related macular degeneration (ARMD), diabetic retinopathy, chronic corneal disease (e.g., corneal dystrophy), optic neuropathy/atrophy (e.g., CNS tumor).
      Vision loss associated with pain: Acute angle closure glaucoma, optic neuritis (may have pain with eye movements), uveitis, endophthalmitis, corneal hydrops (keratoconus).
   Posttraumatic vision loss: Eyelid swelling, corneal irregularity, hyphema, ruptured globe, traumatic cataract, commotio retinae, retinal detachment, retinal or vitreous hemorrhage, lens dislocation, traumatic optic neuropathy, cranial neuropathies, CNS injury, sympathetic ophthalmia (rare).
Distorted vision: Refractive error (including presbyopia, acquired myopia such as from cataract, diabetes, pregnancy, ciliary spasm or ciliary body rotation, medications, retinal detachment surgery), acquired astigmatism (e.g., from anterior segment surgery, periorbital or eyelid edema/mass such as chalazion, orbital trauma), macular disease (e.g., central serous chorioretinopathy, macular edema, ARMD, and others associated with choroidal neovascular membranes (CNVMs), corneal irregularity, intoxication (e.g., ethanol, methanol), pharmacologic (e.g., scopolamine patch), keratoconus, topical eye drops (e.g., miotics, cycloplegics), retinal detachment, migraine (transient), hypotony, CNS abnormality (including papilledema), non-physiologic.
Double vision:
   Monocular (diplopia remains when the uninvolved eye is occluded): Refractive error, incorrect spectacle alignment, corneal opacity or irregularity (including corneal or refractive surgery), cataract, iris defects (e.g., iridectomy), dislocated natural lens or lens implant, macular disease, retinal detachment, CNS causes (rare), non-physiologic.
   Binocular (diplopia eliminated when either eye is occluded):
      Intermittent: Myasthenia gravis, intermittent decompensation of an existing phoria.
      Constant: Isolated sixth, third, or fourth nerve palsy; orbital disease (e.g., thyroid eye disease; idiopathic orbital inflammation [orbital pseudotumor], tumor); cavernous sinus/superior orbital fissure syndrome; status-post ocular surgery (e.g., residual anesthesia, displaced muscle, muscle surgery, restriction from scleral buckle, severe aniseikonia after refractive surgery); status-post trauma (e.g., orbital wall fracture with extraocular muscle entrapment, orbital edema); convergence/divergence insufficiency; internuclear ophthalmoplegia; vertebrobasilar artery insufficiency; other CNS lesions; spectacle problem.
Dry eyes: Burning, dryness, foreign body sensation, mildly to moderately decreased vision, excess tearing. Often exacerbated by smoke, wind, heat, low humidity, or prolonged use of the eye (e.g., when working on a computer that results in decreased blink rate). Usually bilateral and chronic (although patients sometimes are seen with recent onset in one eye). Discomfort often out of proportion to clinical signs
Eyelash loss: Trauma, burn, cutaneous neoplasm (e.g., sebaceous gland carcinoma), eyelid infection or inflammation, radiation, chronic skin disease (e.g., alopecia areata), Vogt-Koyanagi-Harada syndrome, thyroid disease, trichotillomania.
Eyelid crusting: Blepharitis, meibomitis, conjunctivitis, canaliculitis, nasolacrimal duct obstruction, dacryocystitis.
Eyelid swelling:
   Associated with inflammation (usually_erythematous): Hordeolum, blepharitis, conjunctivitis, preseptal or orbital cellulitis, trauma, contact dermatitis, herpes simplex or zoster dermatitis, ectropion, corneal abnormality, urticaria or angioedema, blepharochalasis, insect bite, dacryoadenitis, erysipelas, eyelid or lacrimal gland mass, auto-immunities (e.g., discoid lupus, dermatomyositis).
   Non-inflammatory: Chalazion; dermatochalasis; prolapse of orbital fat (retropulsion of the globe increases the prolapse); eyelid or lacrimal gland mass; eyelid laxity; foreign body; cardiac, renal, or thyroid disease; superior vena cava syndrome.
Eyelid twitch: Orbicularis myokymia (related to fatigue, excess caffeine, medication, or stress), corneal or conjunctival irritation (especially from an eyelash, cyst, or foreign body/suture), dry eye, blepharospasm (bilateral), hemifacial spasm, serum electrolyte abnormality, Tourette's, tic douloureux, albinism/congenital glaucoma (photosensitivity), anemia (rare).
Eyelid unable to close (Lagophthalmos): Severe proptosis, ectropion or eyelid laxity, severe chemosis, eyelid scarring, eyelid retractor muscle scarring, seventh cranial nerve palsy, status-post facial cosmetic or reconstructive surgery.
Eyelid bulging (Proptosis).
Eyes jumping (oscillopsia): Acquired nystagmus, internuclear ophthalmoplegia, myasthenia gravis, vestibular function loss, opsoclonus/ocular flutter, superior oblique myokymia, various CNS disorders.
Flashes of light: Retinal break or detachment, posterior vitreous detachment, migraine, rapid eye movements (particularly in darkness), oculodigital stimulation, dysphotopsias caused by intraocular lens, CNS (particularly occipital lobe) disorders, vestibulobasilar artery insufficiency, optic neuropathies, retinitis/uveitis, entoptic phenomena, drug-related, hallucinations, iatrogenic (e.g., post laser photocoagulation).
Foreign body sensation: Dry eye syndrome, blepharitis, conjunctivitis, trichiasis, corneal or conjunctival abnormality (e.g., cyst, corneal abrasion or foreign body, recurrent erosion, superficial punctate keratopathy), contact lens-related problem, episcleritis, pterygium, pinguecula.
Glare: Cataract, pseudophakia, posterior capsular opacity, corneal irregularity or opacity, altered pupillary/iris structure or response, status-post refractive surgery, posterior vitreous detachment, pharmacologic (e.g., atropine).
Halos around lights: Cataract, pseudophakia, posterior capsular opacity, acute angle closure glaucoma or corneal edema from another cause (e.g., aphakic or pseudophakic bullous keratopathy, contact lens overwear), corneal dystrophies, status-post refractive surgery, corneal haziness, discharge, pigment dispersion syndrome, vitreous opacities, drugs (e.g., digitalis, chloroquine).
Itchy eyes: Conjunctivitis (especially allergic, vernal, and viral), blepharitis, dry eye syndrome, topical drug allergy or contact dermatitis, giant papillary conjunctivitis, or contact lens related problems.
Light Sensitivity (photophobia):
   Abnormal eye examination: Corneal abnormality (e.g., abrasion or edema), anterior uveitis, conjunctivitis (mild photophobia), posterior uveitis, scleritis, albinism, total color blindness, aniridia, mydriasis of any etiology (e.g., pharmacologic, traumatic), congenital glaucoma.
   Normal eye examination: Migraine, meningitis, retrobulbar optic neuritis, subarachnoid hemorrhage, trigeminal neuralgia, or lightly pigmented irises.
   Night blindness: Refractive error (especially under-corrected myopia), advanced glaucoma or optic atrophy, small pupil (especially from miotic drops), retinitis pigmentosa, congenital stationary night blindness, status-post pan-retinal photocoagulation, drugs (e.g., phenothiazines, chloroquine, quinine), vitamin A deficiency, gyrate atrophy, choroideremia.
Pain sensation:
   Ocular:
      Mild to moderate: Dry eye syndrome, blepharitis, infectious conjunctivitis, episcleritis, inflamed pinguecula or pterygium, foreign body (corneal or conjunctival), corneal disorder (e.g., superficial punctate keratopathy), superior limbic keratoconjunctivitis, ocular medication toxicity, contact lens-related problems, postoperative, ocular ischemic syndrome, eye strain from uncorrected refractive error (asthenopia).
      Moderate to severe: Corneal disorder (e.g., abrasion, erosion, infiltrate/ulcer/keratitis, chemical injury, ultraviolet burn), trauma, anterior uveitis, scleritis, endophthalmitis, acute angle closure glaucoma
   Periorbital: Trauma, hordeolum, preseptal cellulitis, dacryocystitis, dermatitis (e.g., contact, chemical, varicella zoster, or herpes simplex), referred pain (e.g., dental, sinus), giant cell arteritis, tic douloureux (trigeminal neuralgia).
   Orbital: Sinusitis, trauma, orbital cellulitis, idiopathic orbital inflammatory syndrome, orbital tumor or mass, optic neuritis, acute dacryoadenitis, migraine or cluster headache, diabetic cranial nerve palsy, postinfectious neuralgia (herpetic).
   Asthenopia: Uncorrected refractive error, phoria or tropia, convergence insufficiency, accommodative spasm, pharmacologic (miotics).
Red eye:
   Adnexal causes: Trichiasis, distichiasis, floppy eyelid syndrome, entropion or ectropion, lagophthalmos, blepharitis, meibomitis, acne rosacea, dacryocystitis, canaliculitis.
   Conjunctival causes: Ophthalmia neonatorum in infants, conjunctivitis (bacterial, viral, chemical, allergic, atopic, vernal, medication toxicity), subconjunctival hemorrhage, inflamed pinguecula, superior limbic keratoconjunctivitis, giant papillary conjunctivitis, conjunctival foreign body, symblepharon and associated etiologies (e.g., ocular cicatricial pemphigoid, Stevens-Johnson syndrome, toxic epidermal necrolysis), conjunctival neoplasia.
   Corneal causes: Infectious or inflammatory keratitis, contact lens-related problems, corneal foreign body, recurrent corneal erosion, pterygium, neurotrophic keratopathy, medicamentosa, ultraviolet or chemical burn.
   Other: Trauma, postoperative, dry eye syndrome, endophthalmitis, anterior uveitis, episcleritis, scleritis, pharmacologic (e.g., prostaglandin analogs), angle-closure glaucoma, carotid-cavernous fistula (corkscrew conjunctival vessels), cluster headache.
Spots in front of eyes
   Transient: Migraine.
   Permanent or long standing: Posterior vitreous detachment, intermediate or posterior uveitis, vitreous hemorrhage, vitreous condensations/debris, hyphema/microhyphema, retinal break or detachment, corneal opacity/dystrophy or foreign body
Tearing:
   Adults:
      Pain present: Corneal abnormality (e.g., abrasion, foreign body or rust ring, recurrent erosion, edema), anterior uveitis, eyelash or eyelid disorder (e.g., trichiasis, entropion), conjunctival foreign body, dacryocystitis, dacryoadenitis, canaliculitis, trauma.
      Minimal/no pain: Dry eye syndrome, blepharitis, nasolacrimal duct obstruction, punctal occlusion, lacrimal sac mass, ectropion, conjunctivitis (especially allergic and toxic), crocodile tears (congenital or seventh nerve palsy), emotional state.
   Children: Nasolacrimal duct obstruction, congenital glaucoma, corneal or conjunctival foreign body, or other irritative disorder.

In some embodiments, at an inference step 232, a trained machine learning model of the present technique may be applied to target dataset comprising one or more images and associated datapoints with respect to a target subject, to predict a diagnosis associated with an anterior segment eye condition in the target subject. In some embodiments, the machine learning model may be further configured to assign a confidence score to each prediction which represents the likelihood that the prediction is correct.

In some embodiments, the predicted diagnosis indicates the presence or absence of an anterior segment eye condition in the target subject, wherein the indication is expressed as a binary value.

In some embodiments, the predicted diagnosis indicates a severity level associated with an anterior segment eye condition in the target subject, and wherein the indication is expressed as a value on a scale.

In some embodiments, the predicted diagnosis indicates an anterior segment eye condition in the target subject, selected from a defined set of possible anterior segment eye conditions. In some embodiments, the predicted diagnosis is a differential diagnosis including two or more possible diagnoses, each with an assigned confidence score.

In some embodiments, the target dataset may be acquired, at least in part, from a target subject at a remote location, using, e.g., a software application running on a mobile device, such as smartphone 120 shown in FIG. 1.

In some embodiments, the inference step 232 may be performed locally on the device utilizing local software integrated into the device. In some embodiments, the analysis is performed remotely on a remote system or server, such as system, 100 shown in FIG. 1, after the target dataset is uploaded over a network.

In some embodiments, the software application of the present technique also includes a clinician interface to facilitate evaluation and analysis of the data and images gathered from the patient, and provide treatment recommendations. In some embodiments, an output of the clinician evaluation and analysis phase may be a treatment recommendation.

In some embodiments, inference step 232 may be implemented using a software application of the present technique, which may be executed on a mobile device or another home device. The software application may provide for a structured questionnaire for self-completion by the target subject, to gather the target eye images and associated datapoints. A medical practitioner, such as an ophthalmologist, may provide online real-time, near-real time, or non-direct quality control and guidance to the patient, by reviewing questionnaire responses and/or other data gathering steps, and providing direct feedback.

In another exemplary implementation, inference step 232 may be implemented using a software application of the present technique, which may be executed on a dedicated device, e.g., a dedicated ophthalmic healthcare system. The software application may provide for a structured questionnaire for self-completion by the target subject, to gather the target eye images and associated datapoints. A medical practitioner, such as an ophthalmologist, may provide online real-time, near-real time, or non-direct, quality control and guidance to the patient, by reviewing questionnaire responses and/or other data gathering steps, and providing direct feedback.

In yet another exemplary implementation, inference step 232 may be implemented using a software application of the present technique, which may be executed on a mobile device or another home device. The software application may provide for at least partially guided-completion by the target subject, in the presence and under the supervision of a medical practitioner, such as a healthcare worker, which provides real-time quality control and guidance to the target subject. An ophthalmologist may also provide online real-time, near-real time, or non-direct quality control and guidance to the patient, by reviewing questionnaire responses and/or other data gathering steps, and providing direct feedback.

In a further exemplary implementation, inference step 232 may be implemented using a software application of the present technique, which may be executed on a mobile device or another home device. The software application may provide for a structured questionnaire for self-completion by the target subject, to gather the target eye images and associated datapoints. A medical practitioner, such as a healthcare worker, may provide online real-time, near-real time, or non-direct quality control and guidance to the target subject, by reviewing questionnaire responses and/or other data gathering steps, and providing direct feedback. An ophthalmologist may provide online real-time, near-real time, or non-direct quality control and guidance to the healthcare worker, by reviewing questionnaire responses and/or other data gathering steps, and providing direct feedback to the healthcare worker.

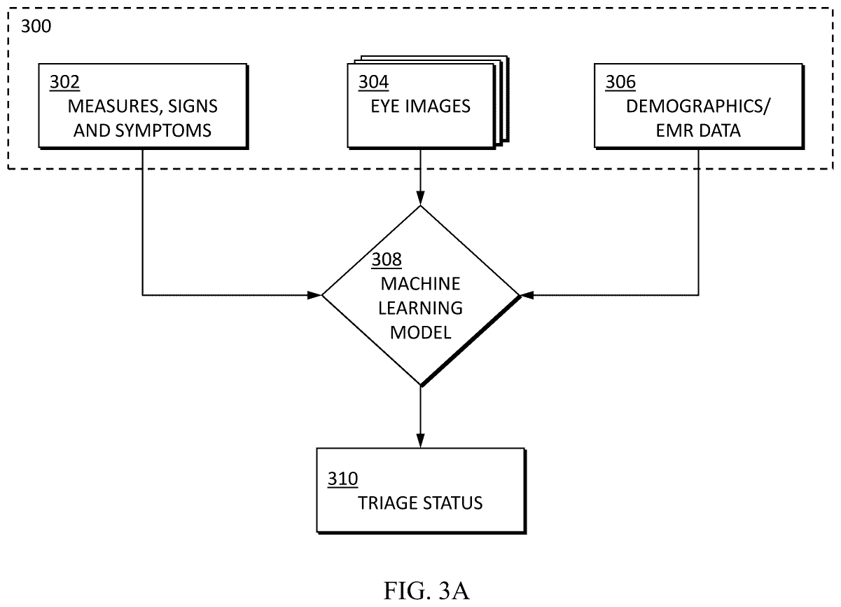

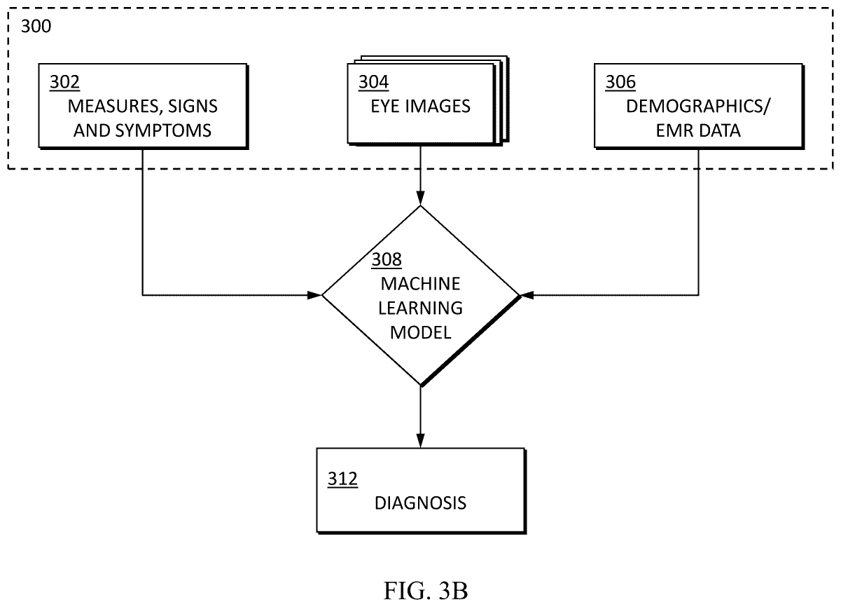

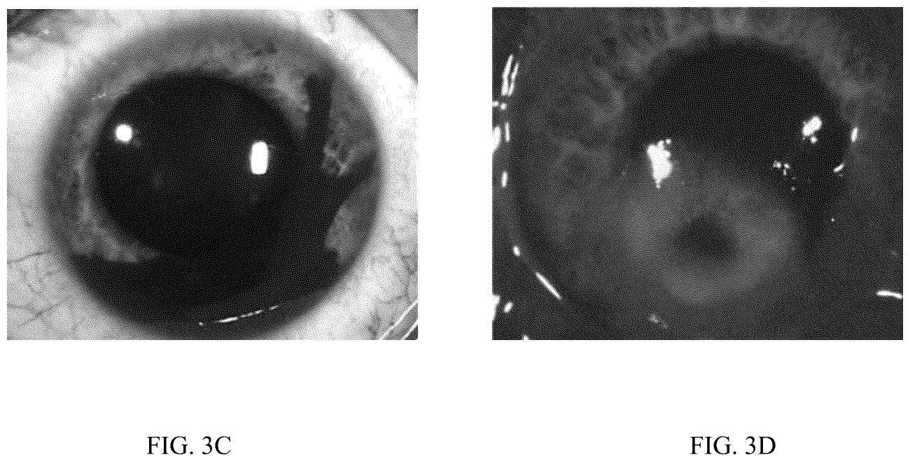

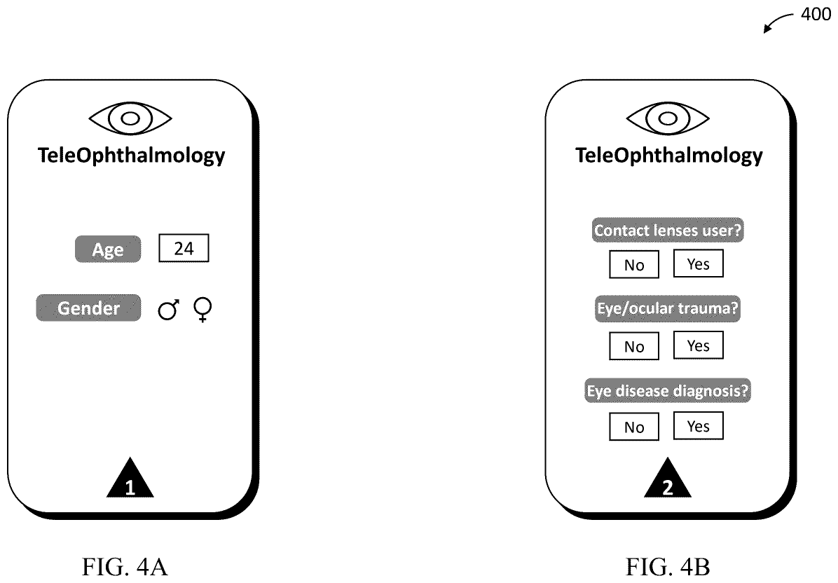

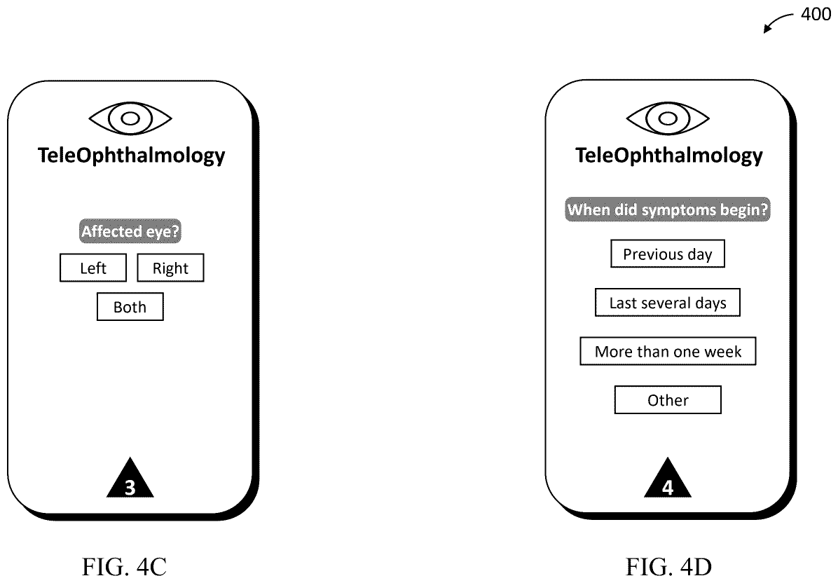

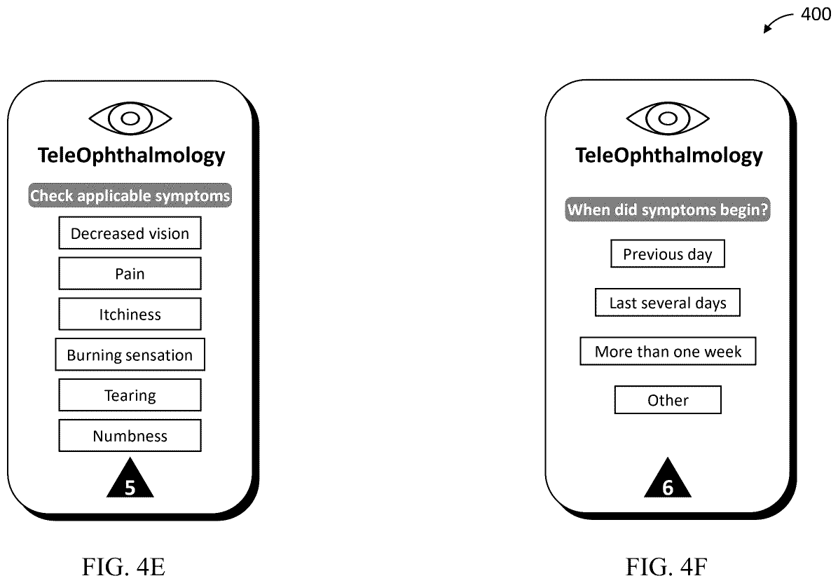

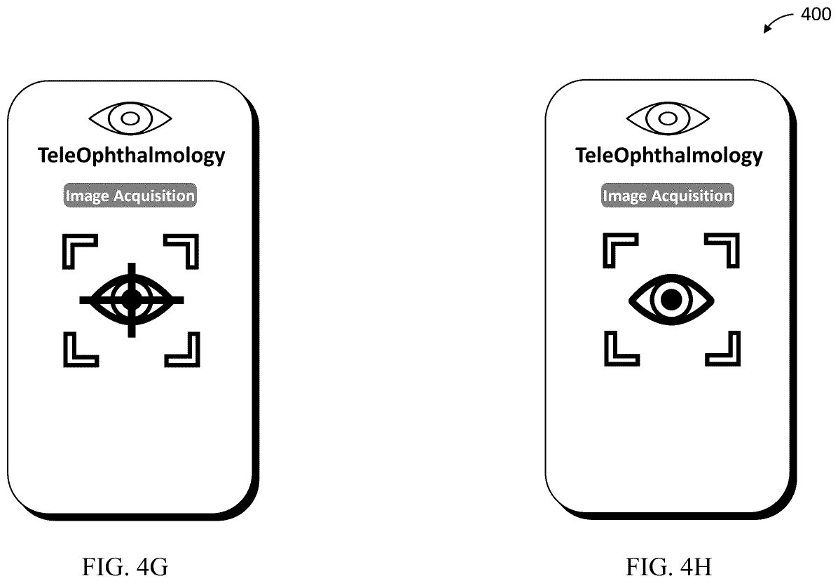

FIGS. 3A and 3B schematically illustrate the process of inference steps 212 in method 200, and 232 in method 220, respectively. As can be seen, a target dataset 300 may be acquired, including a set of eye condition measures, signs, and symptoms 302 associated with a target subject, one or more images 304, and historical medical records (which may be in the form of EMR) 306.

The target dataset 300 may be input into a trained machine learning model 308 for inferencing. Machine learning model 308 may provide, as output, one or more of a triage status 310 (FIG. 3A) or diagnosis of an anterior segment eye condition or disease 312 (FIG. 3B).

For example, a target dataset may include one or more images showing blood pooled behind the cornea, such as the exemplary image shown in FIG. 3C. The target dataset may also include the following symptoms:
pain,
blurred vision,
history of blunt trauma.

In such cases, a possible diagnosis may be traumatic hyphema.

In another example, a target dataset may include one or more images showing white infiltrate on the cornea, such as the exemplary image shown in FIG. 3D. The target dataset may also include the following symptoms:
Red eye,
moderate-to-severe ocular pain,
photophobia,
decreased vision,
discharge,
acute contact lens intolerance pain,
blurred vision, and
history of blunt trauma.

In such cases, a possible diagnosis may be bacterial Keratitis (e.g., with a confidence score of 85%). Less likely diagnoses (e.g., with a confidence score of 15%) may be one of:
Fungal infection,
Acanthamoeba,
herpes simplex virus,
atypical microbacteria,
sterile corneal thinning and ulcers,
staphylococcal hypersensitivity,
sterile corneal infiltrates,
residual corneal foreign body or rust ring, or
topical anesthetic abuse.

Treatment recommendations in such case may include:
Taking corneal scrapings and culture,
applying fluoroquinolone drops 6 times a day,
referral to Emergency Room.

FIGS. 4A-4H illustrate an exemplary user interface 400 of a software application for remote data gathering from a target subject. In some embodiments, interface 400 shown in FIGS. 4A-4F is configured to guide the patient through a structured questionnaire to gather demographic information about the patient, as well as self-reported symptoms. In some embodiments, interface 400 is further configured to guide the patient through one or more standardized visual acuity tests. As shown in FIGS. 4G-4H, interface 400 further includes a facility for guiding the patient through acquiring and uploading one or more images of the patient's eye, e.g., using a camera of the mobile device. In some embodiments, natural language processing (NLP) and artificial intelligence (AI) tools, such as chatbots and the like, may be used for review and quality control of questionnaire responses and other data submitted by a patient. For example, such tools may detect incomplete or non-responsive answers. In some embodiments, NLP and AI tools may further be used for review and quality control of diagnoses and treatment recommendations by ophthalmologists. In some embodiments, decision trees algorithms may be used to guide a patient through different branches of questions trees and supplemental data requests, for example, based on previous answers by the patient. Likewise, NLP and AI tools may be used to select follow-up questions and prompts, based on previous answers and data provided by the patient.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, a field-programmable gate array (FPGA), or a programmable logic array (PLA) may execute the computer readable program instructions by utilizing state information of the computer readable program instructions to personalize the electronic circuitry, in order to perform aspects of the present invention. In some embodiments, electronic circuitry including, for example, an application-specific integrated circuit (ASIC), may be incorporate the computer readable program instructions already at time of fabrication, such that the ASIC is configured to execute these instructions without programming.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the description and claims, each of the terms “substantially,” “essentially,” and forms thereof, when describing a numerical value, means up to a 20% deviation (namely, ±20%) from that value. Similarly, when such a term describes a numerical range, it means up to a 20% broader range—10% over that explicit range and 10% below it).

In the description, any given numerical range should be considered to have specifically disclosed all the possible subranges as well as individual numerical values within that range, such that each such subrange and individual numerical value constitutes an embodiment of the invention. This applies regardless of the breadth of the range. For example, description of a range of integers from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 4, and 6. Similarly, description of a range of fractions, for example from 0.6 to 1.1, should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example 0.7, 1, and 1.1.

The descriptions of the various embodiments of the present invention have been presented for purposes of illustration, but are not intended to be exhaustive or limited to the explicit descriptions. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise,” “include,” and “have,” as well as forms thereof, are not necessarily limited to members in a list with which the words may be associated.

Where there are inconsistencies between the description and any document incorporated by reference or otherwise relied upon, it is intended that the present description controls.

1. A system comprising:
at least one hardware processor; and
a non-transitory computer-readable storage medium having stored thereon program instructions, the program instructions executable by the at least one hardware processor to:
receive, as input, a set of images of eyes of a cohort of subjects, and a plurality of associated datapoints with respect to each of the subjects in the cohort, wherein at least some of said subjects in said cohort are associated with a diagnosis of a specific eye condition,
extract, with respect to each of said subjects, a set of features representing said images and said datapoints associated with said subject,
at a training stage, train a machine learning model on a training dataset comprising:
(i) all of said sets of features, and
(ii) labels indicating a diagnosis of an eye condition associated with each of said subjects, and
at an inference stage, apply said trained machine learning model to a target dataset comprising one or more images and associated datapoints with respect to a target subject, to output a diagnosis of an eye condition in said target subject.

2. The system of claim 1, wherein said specific eye condition is an anterior segment eye condition.

3. The system of claim 1, wherein said images are annotated to indicate anatomical and pathological eye features represented in said images, and wherein said annotations are in the form of one of: an exact outline of each of said anatomical and pathological features, or a bounding box enclosing said each of anatomical and pathological features.

4. (canceled)

5. The system of claim 1, wherein said datapoints comprise, with respect to each of said subjects, at least one of the following categories of datapoints:
(i) demographic information datapoints;
(ii) medical history datapoints; and
(iii) eye condition signs and symptoms datapoints.

6. The system of claim 1, wherein said labels represent, with respect to each of said subjects, binary values indicating the presence or absence of an anterior segment eye condition, and wherein said diagnosis is expressed as a binary value indicating the presence or absence of an anterior segment eye condition in said target subject.

7. The system of claim 1, wherein said labels represent, with respect to each of said subjects, values on a scale indicating a severity level associated with an anterior segment eye condition, and wherein said diagnosis is expressed as a value on a scale indicating a severity level associated with an anterior segment eye condition in said target subject

8. (canceled)

9. The system of claim 1, wherein said images are acquired using a mobile device camera.

10. A computer-implemented method comprising:
receiving, as input, a set of images of eyes of a cohort of subjects, and a plurality of associated datapoints with respect to each of the subjects in the cohort, wherein at least some of said subjects in said cohort are associated with a diagnosis of a specific eye condition;
extracting, with respect to each of said subjects, a set of features representing said images and said datapoints associated with said subject;
at a training stage, training a machine learning model on a training dataset comprising:
(i) all of said sets of features, and
(ii) labels indicating a diagnosis of an eye condition associated with each of said subjects; and
at an inference stage, applying said trained machine learning model to a target dataset comprising one or more images and associated datapoints with respect to a target subject, to output a diagnosis of an eye condition in said target subject.

11. The computer-implemented method of claim 10, wherein said specific eye condition is an anterior segment eye condition.

12. The computer-implemented method of claim 10, wherein said images are annotated to indicate anatomical and pathological eye features represented in said images, and wherein said annotations are in the form of one of: an exact outline of each of said anatomical and pathological features, or a bounding box enclosing each of said anatomical and pathological features.

13. (canceled)

14. The computer-implemented method of claim 10, wherein said datapoints comprise, with respect to each of said subjects, at least one of the following categories of datapoints:
(i) demographic information datapoints;
(ii) medical history datapoints; and
(iii) eye condition signs and symptoms datapoints.

15. The computer-implemented method of claim 10, wherein said labels represent, with respect to each of said subjects, binary values indicating the presence or absence of an anterior segment eye condition, and wherein said diagnosis is expressed as a binary value indicating the presence or absence of an anterior segment eye condition in said target subject.

16. The computer-implemented method of claim 10, wherein said labels represent, with respect to each of said subjects, values on a scale indicating a severity level associated with an anterior segment eye condition, and wherein said diagnosis is expressed as a value on a scale indicating a severity level associated with an anterior segment eye condition in said target subject.

17. (canceled)

18. The computer-implemented method of claim 10, wherein said images are acquired using a mobile device camera.

19. A computer program product comprising a non-transitory computer-readable storage medium having program instructions embodied therewith, the program instructions executable by at least one hardware processor to:
receive, as input, a set of images of eyes of a cohort of subjects, and a plurality of associated datapoints with respect to each of the subjects in the cohort, wherein at least some of said subjects in said cohort are associated with a diagnosis of a specific eye condition;
extract, with respect to each of said subjects, a set of features representing said images and said datapoints associated with said subject;
at a training stage, train a machine learning model on a training dataset comprising:
(i) all of said sets of features, and
(ii) labels indicating a diagnosis of an eye condition associated with each of said subjects; and
at an inference stage, apply said trained machine learning model to a target dataset comprising one or more images and associated datapoints with respect to a target subject, to output a diagnosis of an eye condition in said target subject.

20. The computer program product of claim 19, wherein said specific eye condition is an anterior segment eye condition.

21. The computer program product of claim 19, wherein said images are annotated to indicate anatomical and pathological eye features represented in said images, and wherein said annotations are in the form of one of: an exact outline of each of said anatomical and pathological features, or a bounding box enclosing each of said anatomical and pathological features.

22. (canceled)

23. The computer program product of claim 19, wherein said datapoints comprise, with respect to each of said subjects, at least one of the following categories of datapoints:
(i) demographic information datapoints;
(ii) medical history datapoints; and
(iii) eye condition signs and symptoms datapoints.

24. The computer program product of claim 19, wherein said labels represent, with respect to each of said subjects, binary values indicating the presence or absence of an anterior segment eye condition, and wherein said diagnosis is expressed as a binary value indicating the presence or absence of an anterior segment eye condition in said target subject, or wherein said labels represent, with respect to each of said subjects, values on a scale indicating a severity level associated with an anterior segment eye condition, and wherein said diagnosis is expressed as a value on a scale indicating a severity level associated with an anterior segment eye condition in said target subject.

25. (canceled)

26. (canceled)

27. The computer program product of claim 19, wherein said images are acquired using a mobile device camera is associated with a confidence score which represents the likelihood that the prediction is correct.

28-48. (canceled)
